(19) United States
(12) Patent Application Publication
(10) Pub. No.: US 2011/0092853 A1
(43) Pub. Date: Apr. 21, 2011

(54) INTERVENTION DEVICE FOR COLLECTING BIOLOGICAL MATERIAL AND METHOD FOR THE PRODUCTION THEREOF

(76) Inventors: Karsten Hiltawsky, Schwerte (DE); Sven Meyburg, Erlangen (DE); Daniel Sickert, Munchen (DE)

(21) Appl. No.: 12/996,121

(22) PCT Filed: May 29, 2009
(86) PCT No.: PCT/EP2009/056596

§ 371 Date: Dec. 3, 2010

(30) Foreign Application Priority Data
Jun. 6, 2008 (DE) 10 2008 027 095.4

(51) Int. Cl. A61B 5/15; G01N 1/28

(52) U.S. Cl. 600/582; 427/2.13

(57) ABSTRACT

An intervention device is disclosed for collecting biological material comprises a surface with a coating covering the surface at least in part. In at least one embodiment, capture molecules, by which the biological material can be bound, are immobilized on the coating. The capture molecules are distributed stochastically on the coating.

### PRIORITY STATEMENT

This application is the national phase under 35 U.S.C. §371 of PCT International Application No. PCT/EP2009/056596 which has an International filing date of May 29, 2009, which designates the United States of America, and which claims priority on German patent application number DE 10 2008 027 095.4 filed Jun. 6, 2008, the entire contents of each of which are hereby incorporated herein by reference.

### FIELD

At least one embodiment of the present invention generally relates to an intervention device for collecting biological material, for example to one comprising a surface having a coating which at least partly covers the surface, wherein capture molecules via which the biological material is bindable are immobilized on the coating. At least one embodiment also generally relates to a method for producing the intervention device.

### BACKGROUND

For a multiplicity of medical problems, it is necessary to obtain information about the state of the patient at an early stage. By way of example, a patient with a malignant cancer passes through multiple disease stages depending on the size and on the growth of the tumor. Characteristic of a malignant tumor is, in particular, its unregulated and progressive invasive growth, such that the original boundary of the organ is exceeded at a certain point. This behavior typically leads, in a subsequent disease phase, to the formation of metastases, i.e., to new tumor tissue in parts of the body other than the original location. These metastases are induced by way of circulating tumor cells. These cells detach from the original tumor and circulate in the blood through the body of the patient, and this can lead to the formation of metastases. The number of circulating tumor cells in the blood of the patient allows a statement to be made about the course and state of the disease. For example, if the number of circulating tumor cells decreases during therapy, this is an indication of successful treatment.

Analogously, the detection of circulating endothelial cells is used in the diagnosis of vascular diseases.

In principle, circulating tumor cells can be detected in the blood sample from a patient when they are present at a sufficient number. This is used in particular in later disease stages for the purpose of monitoring the course of the therapy. A known method (CellSearch from Veridex) for determining the number of circulating tumor cells makes use of a blood sample having a maximum volume of 7.5 ml. By utilizing magnetic particles which carry the antibody anti-EpCAM, the tumor cells are bound from the blood sample and, as a result, accumulated. Advantage is taken of the fact that most circulating tumor cells have the EpCAM antigen on their cell surface. The accumulated tumor cells can be isolated, stained, and examined by microscopy in further procedural steps. The method described is, however, only suitable for monitoring the course of the disease and therapy. Owing to the small blood volume which can be used, the probability of enough circulating tumor cells being present in the blood sample in early disease stages is low.

In the treatment of sepsis or septic shock, rapid treatment with antibiotic medicaments is crucial for successful treatment. The therapy should, if possible, begin within an hour after the diagnosis. It is, however, not possible within this time and with currently known methods to determine the pathogen and the resistance thereof. Therefore, a broad-acting antibiotic is generally used. This, in turn, results in an increased risk of the emergence of resistances, an increased toxicity, and increased costs for the treatment. The time required for generating an antibiogram is generally a few days, and so only after this period is it possible to switch to a more specific therapy with antibiotics. Here, it is desirable to make it possible to identify the pathogens and the resistances thereof within a period of between one and two hours.

Various methods are known for identifying pathogens and resistances thereof. The pathogens are, for example, detected in a blood culture. However, setting up and evaluating a blood culture takes a number of days, and a result will therefore in this case come too late for immediate therapeutic decisions. Alternatively, it is possible to use PCR-based diagnostics to identify the pathogen. A blood sample from the patient has to be prepared in a laborious process in which about 10 to 100 pathogens have to be isolated from a blood volume of several milliliters. In turn, DNA is obtained from the pathogens and amplified by means of PCR. By detecting and identifying the DNA of the pathogen, the pathogen itself can be identified. Thus, for example, results can be achieved in six to eight hours from 3 ml of blood and the therapy can thus be specified shortly thereafter (e.g., by means of the LightCycler SeptiFast Test from Roche).

Both in the case of the circulating tumor cells and in the case of the pathogens, it is desirable to identify the biological material concerned (tumor cells, pathogens, etc.) as soon as possible after the diagnosis.

A diagnostic nanosensor is known from EP 1 811 302 A1. This nanosensor consists of, for example, a catheter or stent which has a nanostructured surface. Capture molecules are immobilized on this surface, and by means of these molecules, disease pathogens, for example, can be accumulated and detected directly in the blood of the patient on the surface of the catheter. To produce the nanostructured surface, spheric nanoparticles are applied to the surface of the catheter and optionally fused with one another. The nanoparticles act as a mask for the subsequent vapor deposition of a metal or semiconductor layer. This results in the surface of the catheter having discrete nanoislands whose distance and size are determined by the size of the nanoparticles.

### SUMMARY

At least one embodiment of the present invention provides a simplified intervention device for collecting biological material and a method for the production thereof.

According to one embodiment of the invention, an intervention device for collecting biological material is provided, comprising a surface having a coating which at least partly covers said surface, wherein capture molecules via which the biological material is bindable are immobilized on the coating. The capture molecules are distributed stochastically on the coating. The mobilization on the surface of capture molecules directed against the biological material makes it possible to fish out the biological material from the bloodstream of a patient in an interventional manner. “Stochastic” is intended to be understood here as meaning a random, statistical distribution of the capture molecules which is not subject to any geometric constraints or is subject to only irrelevant ones. “Biological material” is intended to be understood as meaning all types of substances which can be or have to be removed from the human body for analytical purposes. These include, in particular, bacteria, viruses, tumor cells, molecules, and cells or cell constituents in general.

The intervention device is, in some aspects, designed to be comparable to the known prior art. Here, however, the focus is on a stochastic distribution of the capture molecules on the coating. In contrast, the coating of the known intervention devices in the prior art is provided with a nanostructure, and a stochastic distribution of the capture molecules is therefore not possible. The coating method used in the prior art generates discrete nanoislands whose distance varies depending on how the coating was actually carried out. In the intermediate space between the nanoislands, no capture molecules can be immobilized. The present invention is based on the finding that such a discrete distribution of the capture molecules on the discrete nanoislands is rather disadvantageous for the binding of biological material (cells for example). This results from the random distribution of receptor molecules on the biological material.

Owing to the likewise random distribution of the capture molecules on the coating, many of the receptor molecules of the biological material will find a binding partner on the intervention device. This results, firstly, in an increase in the probability of binding and, secondly, in an improvement in the strength of binding of an individual specimen of the biological material to the intervention device. This is, in particular, important for the investigations mentioned at the beginning, i.e., in general when the bloodstream of a patient is screened for only a few specimens of the biological material over a comparatively long period using the intervention device.

In an advantageous embodiment of the invention, the coating comprises at least one closed area which is of a greater magnitude than the biological material to be accumulated. This makes sure that the capture molecules can be stochastically distributed at least on the scale of the contact area between the intervention device and a specimen of the biological material, and a high probability of capture is therefore ensured. A complete coating of the intervention device is therefore not absolutely necessary. A multiplicity of capture molecules is immobilized on the completely covered area.

Alternatively, it is possible to coat the intervention device over the entire surface at least of the part which is actually introduced into the bloodstream of a patient.

Here, even a stochastic distribution of the capture molecules on the entire coating is possible, and the probability of capture is therefore further increased. In addition, a coating of the entire surface is especially simple to achieve.

In an advantageous embodiment of the invention, the coating consists of a metal. Use can be made of, for example, gold, silver, or platinum. Metal layers of this kind are, firstly, easily producible by, for example, vapor deposition or sputtering. Secondly, the capture molecules can be easily immobilized on metal layers of this kind by means of known methods.

In alternative embodiments of the invention, the coating can be produced from a semiconductor material or from an organic polymer.

One advantageous embodiment of the intervention device is formed such that it is introducible into the bloodstream of a patient via an indwelling venous cannula. For a, for example, wire-shaped intervention device, a simple coating with the materials mentioned is possible. In addition, a wire or catheter can be introduced into the bloodstream of the patient via an indwelling venous cannula over a defined period so that the biological material, if present in the bloodstream, can form a bond with the capture molecules of the intervention device. This makes a subsequent detection in vitro possible.

The method-based object is achieved by providing a method for producing an intervention device, comprising the following procedural steps:
providing a support having a surface,
applying the coating onto the surface of the support,
applying the capture molecules onto the coating such that a stochastic distribution on the coating is achieved.

The support can include, for example, a catheter or a wire which is suitable for introduction into the bloodstream of a patient. Even when applying the coating onto the surface of the support, care should be taken that a stochastic distribution is enabled for the subsequent immobilization of the capture molecules. Regular, small structures of the coating on the surface of the support are to be avoided so that the capture molecules can distribute randomly on the coating.

In an advantageous embodiment of the method, the coating is carried out over the entire surface at least of one part of the intervention device, which part is to be introduced into the bloodstream of the patient. Thus, this makes sure that the capture molecules can distribute stochastically on the coating during their application.

In an alternative embodiment of the method, the application generates an area which is of a greater magnitude than the biological material to be accumulated. Thus, this makes sure that, even with a coating that does not cover the entire surface, the capture molecules can distribute stochastically at least on a size scale which is greater than the biological material to be accumulated.

### BRIEF DESCRIPTION OF THE DRAWINGS

Further advantages and embodiments of the invention are revealed in the example embodiments described hereinafter in conjunction with the figures in which

FIG. 1 shows a known embodiment of an intervention device, and

FIG. 2 shows preferably an embodiment of the invention.

### DETAILED DESCRIPTION OF THE EXAMPLE EMBODIMENTS

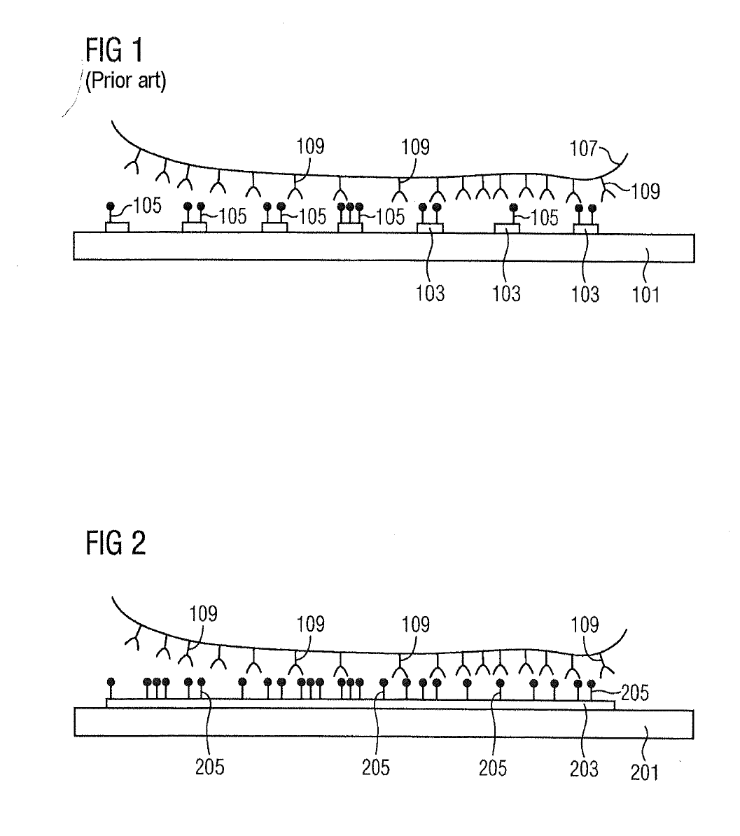

FIG. 1 depicts a known embodiment of an intervention device. On a support 101, there is applied a multiplicity of nanoislands 103 which consist of, for example, gold. The nanoislands 103 are discretely and regularly spaced. Capture molecules 105 are immobilized on the nanoislands 103. Owing to the size of the nanoislands 103, only a few specimens of the capture molecules 105 fit onto an individual nanoisland 103. A section of a cell 107 is depicted above the support 101. Receptor molecules 109 are distributed on the surface of the cell 107. The capture molecules 105 are chosen such that they can form bonds with the receptor molecules 109 of the cell 107. Thus, the cell 107 can be bound to the support 101 when the support 101 is in the bloodstream of a patient. Downstream detection of the cell in vitro is thus possible. Owing to the spacing of the nanoislands 103, a multiplicity of receptor molecules 109, however, does not find a binding partner among the capture molecules 105, and therefore, firstly, the probability of capture is not especially high and, secondly, a possibly established bond is weak owing to the few binding partners.

FIG. 2 depicts schematically a preferred example embodiment of the invention. A coating 203 is applied on a support 201. Capture molecules 205 are immobilized on the coating 203. The capture molecules 205 are distributed stochastically on the coating 203. The cell 107 with its receptor molecules 109 is depicted above the support 201. Owing to the stochastic distribution of the capture molecules 105, distinctly more of the receptor molecules 1.09 find a binding partner among the capture molecules 105. As a result, the probability of capture is increased and the bonds between the support 201 and, once captured, a specimen of the cell 107 are strengthened.

In the example embodiment in FIG. 2, it is possible to use different coating geometries. Firstly, a large part of the support 201 can be coated for example. Preferably, it is this part of the support which is later used in the bloodstream of the patient to capture the cells or other biological material. It is alternatively possible for the coating on the support 201 to include islands, similar to the prior art. However, it then has to be made sure that either the islands, if small, are distributed stochastically on the support 201 or the individual islands are sufficiently large for a stochastic distribution of a sufficient number of capture molecules 205 to be possible at least on the scale of a contact area between the coating 203 and the cell 107.

In a production method for a corresponding wire or catheter, the wire or catheter can, for example, be covered homogeneously with a gold layer. Various possibilities are known for the subsequent immobilization of the capture molecules. For example, capture molecules can be bonded to a gold surface via a thiol bond. Alternatively, it is possible to silanize the surface and, thus, to allow bonding of the capture molecules to the surface. The actual bonding of the capture molecules to the functional features of the surface is carried out by, for example, immersing the wire into a solution containing capture molecules.

The gold layer can be generated by, for example, vapor deposition or sputtering. To produce irregular nanoislands, a tempering step, for example, can be carried out at an appropriately selected temperature. Alternatively, colloidal gold particles can be applied, which are then melted by tempering to form a gold layer or stochastically distributed gold islands. As alternatives to gold, use can also be made of, for example, silver or platinum.

Instead of the use of metals, organic polymers or semiconductors, such as silicon for example, can also be used for coating the catheter surface.

In a further embodiment of the invention, the wire or catheter has at least one thickened section, advantageously at least two thickened sections. The coating having the capture molecules immobilized thereon is located between the thickened sections. This offers a certain protection for the captured cells or pathogens when taking out the wire or catheter from the vein and the indwelling venous cannula of the patient. An accidental stripping off of the captured cells or pathogens is thus effectively prevented. The thickened sections can, for example, be achieved by applying plastic rings onto the wire or catheter. Alternatively, an appropriate shaping of a body forming the wire or catheter is possible.

With the described embodiments of the invention, circulating tumor or endothelial cells or pathogens for example can be accumulated in vivo from the bloodstream of a patient. For this purpose, the wire or catheter having the coating and the corresponding capture molecules, which are antibodies or antibody fragments for example, is introduced into the bloodstream via an indwelling venous cannula or a comparable device. Here, the forward end of the wire or catheter is pushed forward into the blood vessel beyond the length of the indwelling cannula. The tumor cells or pathogens circulating in the blood then pass the catheter end located in the bloodstream and bind to the capture molecules on the coated surface with a certain probability. After a set period of time after which it is certain that enough cells or pathogens for reliable detection have bound, the catheter is removed from the bloodstream. Subsequently, the accumulated cells or pathogens can be detected with the aid of known methods of in vitro diagnostics. The cells or pathogens can, for example with trypsin treatment, be transferred from the catheter into an aqueous solution and thus further processed. In the case of the circulating tumor cells, it is possible for example, as part of further analysis, to carry out staining of the cells with subsequent examination by microscopy, gene expression analysis, or analysis of microRNAs to classify the tumor type. In contrast, pathogens can, for example, be detected with PCR-based methods or via a culture.

An advantage of the described method is that virtually the entire blood volume of a patient can be screened for cells or pathogens. In contrast to known in vitro methods in which it was possible to use only a small defined blood volume, the use of the described devices increases the accuracy of detection. Especially in the case of pathogen diagnostics and in the case of circulating tumor or endothelial cells, the corresponding pathogens or cells can be detected at earlier disease stages than has been the case using known methods. Furthermore, the sample preparation can be carried out more quickly compared with known methods. The antibodies used to bind tumor cells can be, for example, anti-EpCAM molecules.

In addition, by determining the detected cell type, clues to the origin of the still inconspicuous tumor can be found. Thus, for example, the capture of breast cells can provide an indication that, in the breast, a tumor is growing which may morphologically not be particularly conspicuous yet. This represents, for subsequent imaging investigations, an important instrument for increasing the efficiency.

Example embodiments being thus described, it will be obvious that the same may be varied in many ways. Such variations are not to be regarded as a departure from the spirit and scope of the present invention, and all such modifications as would be obvious to one skilled in the art are intended to be included within the scope of the following claims.

1. An intervention device for collecting biological material, comprising:
a surface including a coating which at least partly covers said surface, wherein capture molecules, via which the biological material is bindable, are immobilized on the coating, the capture molecules being distributed stochastically on the coating.

2. The intervention device as claimed in claim 1, wherein the coating comprises at least one closed area which is of a relatively greater magnitude than the biological material to be accumulated.

3. The intervention device as claimed in claim 1, wherein a multiplicity of the capture molecules is immobilized on the at least one closed area.

4. The intervention device as claimed in claim 1, wherein the coating consists of a metal.

5. The intervention device as claimed in claim 4, wherein the metal is gold, silver, or platinum.

6. The intervention device as claimed in claim 1, wherein the coating consists of a semiconductor material,

7. The intervention device as claimed in claim 1, wherein the coating consists of an organic polymer.

8. The intervention device as claimed in claim 1, wherein the capture molecules are directed against a receptor molecule of the biological material.

9. The intervention device as claimed in claim 8, wherein the capture molecules are antibodies.

10. The intervention device as claimed in claim 1, wherein capture molecules are coupled to the coating via a thiol bond.

11. The intervention device as claimed in claim 1, wherein the intervention device is aimed such that it is introducible into the bloodstream of a patient via an indwelling venous cannula.

12. The intervention device as claimed in claim 11, wherein the intervention device is wire-shaped.

13. A method for producing an intervention device for collection of biological material, comprising:
providing a support including a surface;
applying a coating onto the surface of the support; and
applying capture molecules, via which the biological material is bindable, onto the coating such that a stochastic distribution of the capture molecules on the coating is achieved.

14. The method as claimed in claim 13, wherein the application of the coating generates an area which is of a relatively greater magnitude than the biological material to be accumulated.

15. The method as claimed in claim 13, wherein the application of the coating is carried out over the entire surface of at least one part of the support.

16. The method as claimed in claim 13, wherein the application of the coating comprises:
applying a metal layer; and
tempering the metal layer under conditions which allow the formation of metal islands having a stochastic size distribution.

17. The method as claimed in claim 13, wherein the application of the coating comprises:
applying colloidal metal particles; and
tempering the metal particles under conditions which allow the formation of metal islands having a stochastic size distribution.

18. The method as claimed in claim 13, wherein the application of the capture molecules is carried out by immersing the support into a solution containing capture molecules.

19. The intervention device as claimed in claim 2, wherein the coating consists of a metal.

20. The intervention device as claimed in claim 19, wherein the metal is gold, silver, or platinum.

21. The intervention device as claimed in claim 2, wherein the coating consists of a semiconductor material.

22. The intervention device as claimed in claim 2, wherein the coating consists of an organic polymer.

23. The method as claimed in claim 14, wherein the application of the coating is carried out over the entire surface of at least one part of the support.

24. The method as claimed in claim 14, wherein the application of the coating comprises:
applying a metal layer; and
tempering the metal layer under conditions which allow the formation of metal islands having a stochastic size distribution.

25. The method as claimed in claim 14, wherein the application of the coating comprises:
applying colloidal metal particles; and
tempering the metal particles under conditions which allow the formation of metal islands having a stochastic size distribution.

26. The method as claimed in claim 14, wherein the application of the capture molecules is carried out by immersing the support into a solution containing capture molecules.
